(19) United States
(12) Patent Application Publication
(10) Pub. No.: US 2009/0022808 A1
(43) Pub. Date: Jan. 22, 2009

(54) Coated Hyaluronic Acid Particles

(75) Inventors: Julie A. Champion, Pasadena, CA (US); Samir Mitragotri, Goleta, CA (US); Ahmet Tezel, Goleta, CA (US)

Correspondence Address: ALLERGAN, INC., 2525 DUPONT DRIVE, T2-7H, IRVINE, CA, 92612-1599, US

(73) Assignee: Allergan, Inc.

(21) Appl. No.: 12/124,722

(22) Filed: May 21, 2008

Related U.S. Application Data
(60) Provisional application No. 60/939,659, filed on May 23, 2007.

(51) Int. Cl. A61K 9/14; A61K 31/715

(52) U.S. Cl. 424/491; 424/497; 424/493; 514/54

(57) ABSTRACT

The present invention generally relates to particles comprising hyaluronic acid, wherein the particles are coated or encapsulated with a coating. The coating preferably comprises a polymer, protein, polysaccharide, or combination thereof that decreases the rate of degradation of the hyaluronic acid once the particles are placed in an aqueous environment, such as inside mammalian skin. The compositions of the present invention comprising such coated hyaluronic acid are useful for soft tissue augmentation, and are particularly useful as dermal fillers.

### RELATED APPLICATION

This application is based, and claims priority under 35 U.S.C. § 120 to U.S. Provisional Patent Application No. 60/939,659 filed on May 23, 2007 and which is incorporated herein by reference.

### BACKGROUND OF THE INVENTION

a. Field of the Invention

The invention relates to compositions for soft tissue augmentation, and in particular, to compositions useful as dermal fillers. The compositions of the present invention comprise hyaluronic acid that has been covered or encapsulated by a protective coating that helps decrease the rate of degradation of the hyaluronic acid upon contact with an aqueous environment.

b. Background Art

Hyaluronic acid is a non-sulfated glycosaminoglycan that is distributed widely throughout the human body in connective, epithelial, and neural tissues. Hyaluronic acid is also a major component of skin, where it is involved in tissue repair. As skin ages and is repeatedly exposed to the sun's ultra violet rays, dermal cells decrease their production of hyaluronic acid and increase the rate of its degradation. Likewise, aging skin loses collagen, another natural substance necessary to keep skin youthful and resilient. As shown in FIG. 1A, over time, the loss of hyaluronic acid and collagen causes aging skin to develop lines, wrinkles, and folds.

In the past several years, compositions of hyaluronic acid have been used in cosmetic applications to fill wrinkles, lines, folds, scars, and to enhance dermal tissue, for example, to plump lips. Because hyaluronic acid is natural to the human body, it is a generally well tolerated and fairly low risk skin augmentation product.

Some hyaluronic acid compositions contain particles, or microspheres, of non-crosslinked hyaluronic acid suspended in a gel. As shown in FIG. 1B, the gel is injected just below the surface of the skin, at the site of the wrinkle, line, or fold (or scar or dermal tissue to be enhanced). The hyaluronic acid essentially plumps up the skin from beneath the upper layers of skin. The injected hyaluronic acid is hydrophilic, and over time absorbs water from the surrounding tissue, causing the hyaluronic acid to degrade. Compositions of non-crosslinked hyaluronic acid tend to degrade within a few months after injection and thus require fairly frequent reinjection to maintain their skin augmenting effect.

More recently, compositions of cross-linked hyaluronic acid have been used for dermal augmentation. Some such cross-linked compositions contain fairly large particles, around approximately 2 mm each, of hyaluronic acid suspended in a gel. Others are a fairly uniform gel matrix of hyaluronic acid. Because hyaluronic acid is fairly flexible, these large particles and matrices are still suitable for subcutaneous injection. However, because the hyaluronic acid of these compositions is cross-linked and larger, it takes a longer time to degrade after injection. Some of these cross-linked hyaluronic acid compositions have a longevity and augmenting effect of up to 6 months or even longer after injection. While these compositions have a longer lasting effect, they still generally require reinjection approximately twice a year.

With the desire for longer lasting dermal fillers, some physicians and patients turn to a variety of synthetic products such as polyacrylamide, polyactide, and polytetrafluorethylene. While such dermal fillers last longer, they are not natural to the human body and may cause a variety of adverse reactions. Moreover, such synthetic fillers often result in less natural looking skin augmentation.

It is thus desirable to have a skin composition that is made of a natural product such as hyaluronic acid, but which will last longer after injection and require less frequent reinjection while maintaining desired skin augmentation.

### BRIEF SUMMARY OF THE INVENTION

The present invention relates to compositions comprising hyaluronic acid, wherein the hyaluronic acid has been coated or encapsulated to protect it from degradation during use. One aspect of the present invention relates to compositions for soft tissue augmentation. These compositions contain hyaluronic acid particles that are coated to protect the hyaluronic acid from degradation. The coatings may contain a biodegradable polymer, nondegradable polymer, protein, polysaccharide, or a combination thereof. The coatings may be biocompatible and bioresorbable, and allow the hyaluronic acid to degrade over time. However, the coated hyaluronic acid particles of the present invention degrade more slowly than uncoated particles, thereby increasing the longevity of the hyaluronic acid during use for soft tissue augmentation. In one embodiment of the present invention, these compositions are suitable for subcutaneous injection in a mammal.

The hyaluronic acid used in the present invention may be crosslinked or non-crosslinked. In some embodiments of the present invention, cross-linked hyaluronic acid is preferred.

In one embodiment of the present invention, hyaluronic acid is coated with polylactic-co-glycolic acid. In another embodiment of the present invention, hyaluronic acid is coated with albumin. In yet another embodiment of the present invention, hyaluronic acid is coated with alginate.

In some preferred embodiments of the present invention, the coated hyaluronic acid is generally spherical in shape. In one preferred embodiment, the coated hyaluronic acid is in the shape of microspheres, the microspheres being, on average, approximately 10 μm to approximately 500 μm in diameter.

The present invention further relates to compositions comprising hyaluronic acid particles that are encapsulated in a polymer, protein, polysaccharide, or a combination thereof. The encapsulated hyaluronic acid particles are generally spherical in shape. In one embodiment, the compositions of encapsulated hyaluronic acid are suitable for subcutaneous injection in a mammal.

In one preferred embodiment, the hyaluronic acid particles are encapsulated in a polymer, protein, polysaccharide, or a combination thereof that allows for sustained release of the hyaluronic acid in an aqueous environment. In another preferred embodiment, the encapsulated particles of hyaluronic acid are cross-linked with at least one biocompatible polymer to form a hydrogel. In a further preferred embodiment, the encapsulated particles of hyaluronic acid are cross-linked with polyvinyl alcohol.

Another aspect of the present invention relates to dermal fillers for skin augmentation. The dermal filler comprise particles of hyaluronic acid coated with a biocompatible polymer, protein, or polysaccharide. In one embodiment, the coating is about 10 nm to about 50000 nm thick. In another embodiment, the coated particles are generally spherical and are, on average, approximately 50 μm to approximately 2000 μm in diameter. In yet another embodiment, the hyaluronic acid is a cross-linked hyaluronic acid.

In yet another aspect, the present invention relates to a method for repairing or augmenting soft tissue in mammals. The method comprising the steps of selecting the mammalian soft tissue to be repaired or augmented and placing into the mammal's soft tissue an injectable, bioresorbable composition comprising hyaluronic acid particles. The hyaluronic acid particles of the injected composition are coated in a polymer, protein, or polysaccharide.

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1A depicts a cross section of mammalian skin, showing the epidermal, dermal, and subcutaneous layers, and showing lines, wrinkles, and folds on such the skin.

FIG. 1B depicts the cross section of mammalian skin shown in FIG. 1A, showing injection sites for hyaluronic acid for filling lines, wrinkles, and folds.

FIG. 2 is a magnified image of a hyaluronic acid particle that has been coated with albumin.

FIG. 3 is a magnified image of a hyaluronic acid particle that has been coated with alginate.

FIG. 4A is a magnified image of particles of dry non-crosslinked hyaluronic acid that have been encapsulated in polylactic-co-glycolic acid.

FIG. 4B is a magnified image of the particles of FIG. 4B after 10 days of exposure to an aqueous solution.

FIG. 5 is a magnified image of particles of wet non-crosslinked hyaluronic acid that have been encapsulated in polylactic-co-glycolic acid.

FIG. 6 is a magnified image of particles of crosslinked hyaluronic acid that have been encapsulated in polylactic-co-glycolic acid.

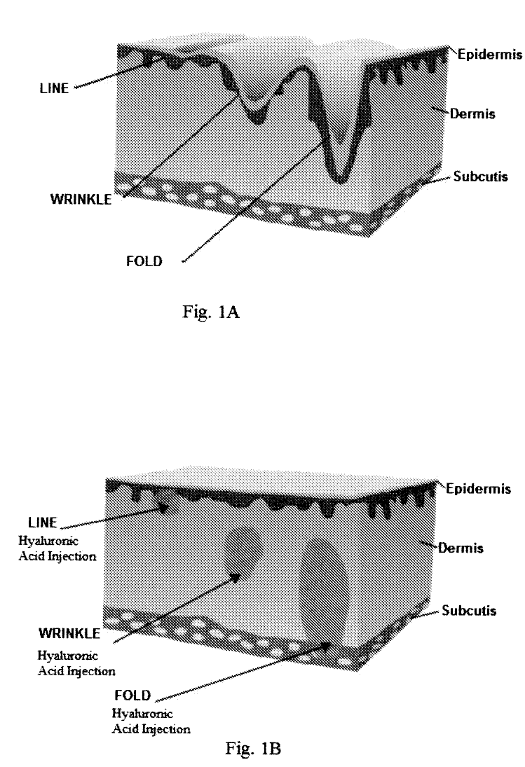

### DETAILED DESCRIPTION OF THE INVENTION

The present invention generally relates to particles comprising hyaluronic acid, wherein the particles are coated or encapsulated with a coating that decreases the rate of degradation of the hyaluronic acid once the particles are placed in an aqueous environment, such as inside mammalian skin. The coated particles of the present invention are intended for use in a composition to repair or augment soft tissue. In one preferred embodiment, the coated particles of the present invention are used in compositions as a dermal filler to fill lines, folds, and wrinkles in skin.

The hyaluronic acid of the present invention may be non-crosslinked, crosslinked, including double crosslinked, single phase or double phase, or a combination of crosslinked and non-crosslinked hyaluronic acid. It may be of any source, including avian or non-animal. The hyaluronic acid may further be combined with other ingredients, such as hypromellose or a bioresorbable polymer, and the combined ingredients may be coated or encapsulated to form the coated particles of the present invention.

The coating may be any type of biocompatible coating material that slows the degradation of hyaluronic acid in an aqueous environment. Preferably, the coating is made of polymers, proteins, polysaccharides, or a combination thereof. Representative synthetic polymers include poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polyurethanes and co-polymers thereof, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof.

Representative proteins include albumin, collagen, gelatin and prolamines like zein. Representative polysaccharides include alginate, cellulose derivatives such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, and cellulose triacetate, and polyhydroxyalkanoates like polyhydroxybutyrate and polyhydroxybutyrate-valerate.

As used herein, “derivatives” include polymers having substitutions, additions of chemical groups and other modifications routinely made by those skilled in the art.

In one preferred embodiment, the coating is made of a polymer, such as polylactide-co-glycolide that allows for sustained release of hyaluronic acid from the particle. The coating may be applied to the hyaluronic acid in any number of ways known to one of skill in the art. The Examples below teach a few non-limiting techniques for creating some of the coated particles of the present invention. The coated particles of the present invention may further be crosslinked into a gel or matrix with a polymer, such as polyvinyl alcohol.

The coating may completely coat, cover, or encapsulate the hyaluronic acid particle, or it may substantially coat the hyaluronic acid particle, sufficient to slow degradation of the hyaluronic acid. In one preferred embodiment, the coating is continuous and substantially uniform.

The coating may also be of any desired thickness, depending on the coating used. For example, a coating of a polymer such as polyethylene glycol or poloxamine may be created physically, e.g., through layer-by-layer deposition, or chemically, e.g., through chemical conjugation, with the hyaluronic acid to make a coating that is only a few nanometers thick.

The preferred size of the coated or encapsulated particles of the present invention varies depending on the type of hyaluronic acid used and the type and thickness of coating. If a very flexible coating is used, the particle size may be larger because the resulting coated particle will be more easily deformable to fit through, for example, a standard needle for subcutaneous injection. If a less flexible coating is applied, a smaller particle size may be necessary. With a smaller particle size, a crosslinked hyaluronic acid may be preferred to further improve the longevity of the coated particle.

For dermal filler embodiments of the present invention, the coated particles must be of a size and flexibility to make them suitable for subcutaneous injection. Such particles should generally be no larger than about 2 mm in diameter. In a further preferred embodiment, the coated particles of the present invention should, on average, be no less than about 10 μm in diameter and no more than about 1000 μm in diameter. In another preferred embodiment, the coated particles are approximately 100 μm to approximately 500 μm in diameter.

The following examples provide further detail regarding some of the embodiments of the present invention.

A. Protein Coatings

The hyaluronic acid of the present invention may be coated with any type of protein. For example, collagen, and/or albumin can be used to coat particles of hyaluronic acid or to create a hyaluronic acid matrix. Preferably, the protein used to coat the hyaluronic acid should be a protein known in the art to be generally readily bioresorbable while allowing for improved in vivo longevity of the coated hyaluronic acid.

As disclosed in Example 1 below, in one preferred embodiment of the present invention, hyaluronic acid is coated with, or encapsulated in, cross-linked albumin to create albumin coated hyaluronic acid microspheres. Albumin is a major plasma protein and is thus biocompatible, biodegradable, and generally non-immunogenic. At the same time, albumin provides a protective coating for hyaluronic acid, giving the coated particles generally better longevity than uncoated particles of hyaluronic acid.

### EXAMPLE 1

A cross-linked hyaluronic acid (Hylaform) was first mixed for 20 minutes at approximately 2000 rpm. The Hylaform was next vortexed with water and Bovine Serum Albumin (BSA) until the BSA was dissolved. The resulting Hylaform/BSA solution was added to mineral oil while stirring at approximately 800 rpm. The mixer speed was next increased to approximately 900 rpm while a solution of 8% gluteraldehyde was added. The solution was stirred for several hours to allow for effective crosslinking of the BSA. The resulting mixture was washed with ethyl ether to remove the mineral oil and the coated particles were washed with water.

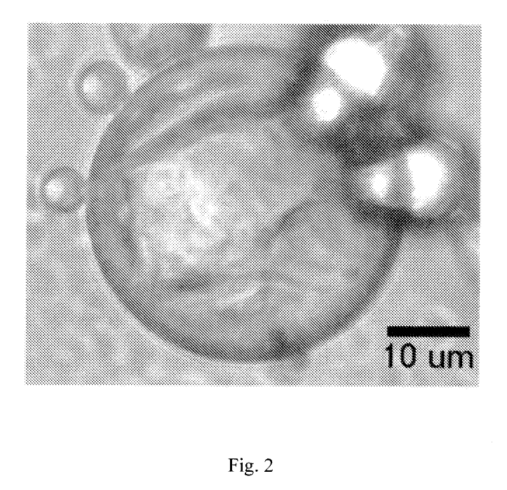

FIG. 2 demonstrates the resulting albumin coated hyaluronic acid particles. The size of the coated particles may be adjusted by adjusting the size of the Hylaform particles used and adjusting the stirring speed during the coating process. The rate of degradation of the albumin coating may be controlled by controlling the cross-linking density of the albumin coating by controlling the gluteraldehyde concentration and length of exposure of the albumin to gluteraldehyde. In one preferred embodiment, the albumin coated particles are approximately 10 μm to approximately 1000 μm in diameter. In a further preferred embodiment, the albumin coated particles are approximately 50 μm to 100 μm in diameter.

B. Polysaccharide Coatings

The hyaluronic acid of the present invention may be coated with any type of polysaccharide. For example, starch, cellulose and derivatives thereof including alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, and cellulose triacetate, and/or alginate can be used to coat particles of hyaluronic acid or to create a hyaluronic acid matrix. Preferably, the polysaccharide used to coat the hyaluronic acid should be a polysaccharide known in the art to be generally readily bioresorbable while allowing for improved in vivo longevity of the coated hyaluronic acid.

As disclosed in Example 2 below, in one preferred embodiment of the present invention, hyaluronic acid is coated with, or encapsulated in, alginate to create alginate coated hyaluronic acid particles. Alginate is a copolymer of glucuronic and mannuronic acid and is readily available. Alginate is hydrophilic, colloidal, and is a non-toxic product that is used in a variety of medical applications.

### EXAMPLE 2

Sodium alginate was dissolved in water, then Hylaform was added by sonication and vortexing. The resulting alginate/HA mixture was added through a small diameter needle to a 0.1M CaCl₂ solution while stirring.

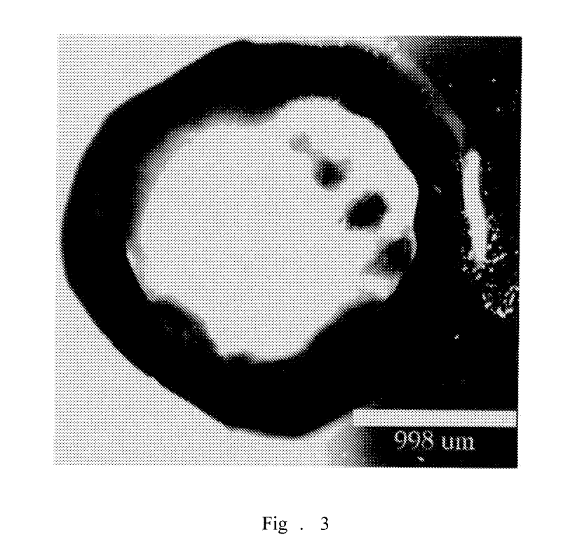

FIG. 3 shows the resulting coated particles. The alginate coated particles are flexible, making them relatively suitable for injection. The alginate coated particles also swell in the presence of water. The size of the coated particles may be adjusted by adjusting the size of the Hylaform particles used and adjusting the concentration of alginate used to adjust the resulting thickness of the coating. In one preferred embodiment, the alginate coated particles are approximately 500 μm to approximately 2000 μm in diameter. In a further preferred embodiment, the albumin coated particles are approximately 500 μm to approximately 1000 μm in diameter. The rate of degradation of the coating may be controlled by adjusting the alginate's cross-linking density and/or by further cross-linking the particles with another protein, such as poly-L-lysine.

C. Polymer Coatings

The hyaluronic acid of the present invention may be coated with any type of bioresorbable or biodegradable polymer, or certain nondegradable polymers. For example, polymers including poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polyurethanes and co-polymers thereof, polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof can be used to coat particles of hyaluronic acid or to create a hyaluronic acid matrix. Such polymers may be coated onto hyaluronic acid through layer-by-layer deposition, chemical conjugation, emulsion, or any variety of coating methods known in the art. The thickness of the coating may be modified to make a very thin coating of only a few nanometers such that large, crosslinked particles of hyaluronic acid may be used and may result in coated particles that are suitable for injection. Or, the coating may be made thicker to improve the longevity of the hyaluronic acid in vivo.

As disclosed in Examples 3, 4, and 5 below, in one preferred embodiment of the present invention, hyaluronic acid is coated with, or encapsulated in, PLGA to create PLGA coated hyaluronic acid microspheres. PLGA is biodegradable and biocompatible, and is approved by the Food and Drug Administration for use in several products. PLGA biodegrades into lactic and glycolic acids which are eliminated by the human body. Additionally, PLGA is not readily water soluble.

### EXAMPLE 3

PLGA (50:50) was dissolved in ethyl formate. Dry, ground, non-crosslinked hyaluronic acid was added to the PLGA solution by vortexing and sonication. The resulting PLGA/HA solution was added to a solution of water and a surfactant, Pluronic F-68, while stirring. The mixture was stirred until most of the ethyl formate evaporated from the mixture.

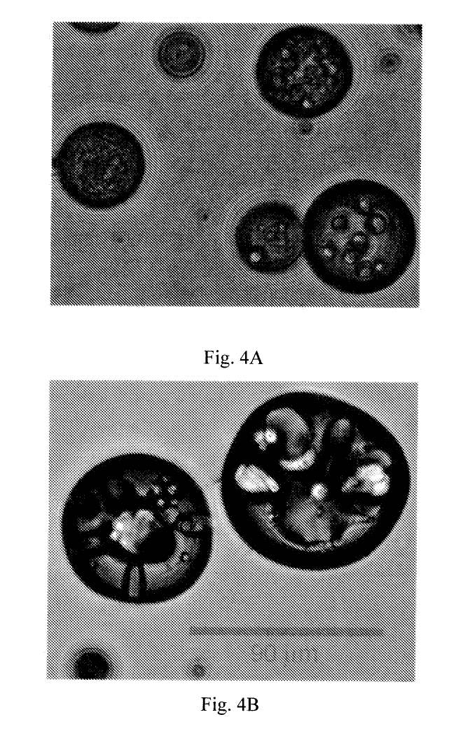

FIG. 4 shows the resulting PLGA coated particles. One advantage of the PLGA coated hyaluronic acid particles of this embodiment is their swelling and slow permeation characteristics. Specifically, PLGA acts like a membrane, allowing slow water permeation into the hyaluronic acid within the coated particles. The hyaluronic acid swells in the presence of water, causing the entire particle to swell. Over time, the PLGA coating biodegrades, allowing hyaluronic acid to be released from the microspheres. The size of the swelling particles may be controlled by controlling the size of the original hyaluronic acid particles and thickness of the PLGA coating. In one preferred embodiment, the PLGA coated particles are approximately 10 μm to approximately 500 μm in diameter. In a further preferred embodiment, the PLGA coated particles are approximately 100 μm to approximately 500 μm in diameter. The longevity of the particle swelling and hyaluronic acid release may be controlled by the thickness of the PLGA coating and the concentration of lactic acid in the PLGA used to create the coating.

### EXAMPLE 4

Non-crosslinked hyaluronic acid was dissolved in water. Separately, PLGA was dissolved in ethyl formate. The solutions were combined and mixed at approximately 2000 rpm for a few minutes. The resulting HA/PLGA emulsion was added to a solution of water and Pluronic F-68 while stirring at approximately 900 rpm. The resulting secondary emulsion was poured into another solution of water and Pluronic F-68 while stirring. Stirring was continued until most of the ethyl formate evaporated.

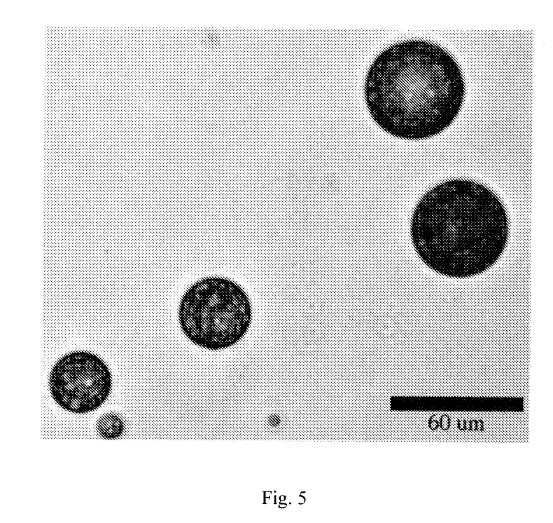

FIG. 5 shows the resulting PLGA particles. The size and degree of polydispersity of these particles may be controlled by controlling stirring parameters. These particles did not exhibit the same swelling characteristics as the PLGA coated particles described in Example 3.

### EXAMPLE 5

PLGA was dissolved in ethyl formate. Dry Hylaform was added to the PLGA solution by vortexing and sonication. The resulting PLGA/HA solution was added to a solution of water and Pluronic F-68 while stirring. The mixture was stirred until most of the ethyl formate evaporated from the mixture.

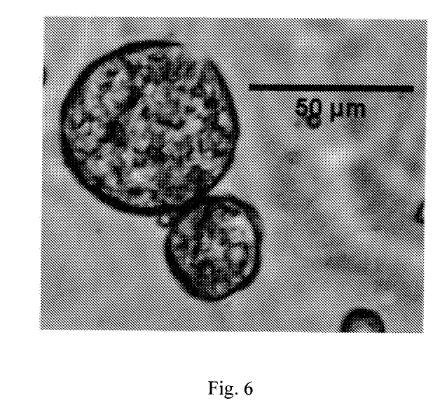

FIG. 6 shows the resulting PLGA coated particles. These particles were generally less uniform and larger than the PLGA coated particles of Example 3. These particles also swelled more quickly and less uniformly than the PLGA coated particles of Example 3.

Although only a few embodiments of this invention have been described above with a certain degree of particularity, those skilled in the art could make numerous alterations to the disclosed embodiments without departing from the spirit or scope of this invention. It is intended that all matter contained in the above description or shown in the accompanying drawings shall be interpreted as illustrative only and not limiting. Changes in detail may be made without departing from the spirit of the invention as defined in the appended claims.

What is claimed is:

1. A composition for soft tissue augmentation, said composition comprising hyaluronic acid coated with a coating to form coated hyaluronic acid, the coating comprising a biodegradable polymer, nondegradable polymer, protein, polysaccharide, or a combination thereof, wherein the composition is suitable for subcutaneous injection in a mammal.

2. The composition of claim 1, wherein the hyaluronic acid is particles of crosslinked hyaluronic acid.

3. The composition of claim 1, wherein the coating is polylactic-co-glycolic acid, albumin, or alginate.

4. The composition of claim 1, wherein the coated hyaluronic acid is in the shape of microspheres being generally spherical in shape.

5. The composition of claim 4, wherein said sustained microspheres are, on average, approximately 10 μm to approximately 2000 μm in diameter.

6. A composition comprising hyaluronic acid particles, wherein the hyaluronic acid particles are encapsulated in a polymer, protein, polysaccharide, or a combination thereof, to form encapsulated hyaluronic acid particles, and wherein the encapsulated hyaluronic acid particles are generally spherical in shape.

7. The composition of claim 6 which is suitable for subcutaneous injection in a mammal.

8. The composition of claim 6, wherein the hyaluronic acid particles are encapsulated in a polymer, protein, or polysaccharide that allows for sustained release of the hyaluronic acid in an aqueous environment.

9. The composition of claim 6, wherein the composition comprises a hydrogel of the encapsulated hyaluronic acid particles cross-linked with at least one biocompatible polymer.

10. The composition of claim 9, wherein the biocompatible polymer is polyvinyl alcohol.

11. A dermal filler for skin augmentation comprising coated particles of hyaluronic acid, said coated particles of hyaluronic acid comprising a coating that decreases the rate of degradation of the hyaluronic acid in an aqueous environment.

12. The dermal filler of claim 11, wherein said coated particles of hyaluronic acid are generally spherical and are, on average, approximately 10 μm to approximately 2000 μm in diameter.

13. The dermal filler of claim 12, wherein the hyaluronic acid of said coated particles of hyaluronic acid is a cross-linked hyaluronic acid.

14. The dermal filler of claim 12, wherein the coating is about 10 nm to 50000 nm thick.

15. A method for repairing or augmenting soft tissue in mammals comprising the steps of: selecting the mammalian soft tissue to be repaired or augmented and placing into the mammal's soft tissue an injectable, bioresorbable composition comprising hyaluronic acid particles coated in a polymer, protein, or polysaccharide.
